(19) United States
(12) Patent Application Publication
(10) Pub. No.: US 2019/0231587 A1
(43) Pub. Date: Aug. 1, 2019

(54) Applicator Device

(71) Applicant: CONTINE CORPORATION, New London, CT (US)

(72) Inventor: Robert M Spitz, New London, CT (US)

(21) Appl. No.: 16/323,733

(22) PCT Filed: Aug. 8, 2017
(86) PCT No.: PCT/US2017/045840

§ 371 Date: Feb. 6, 2019

Related U.S. Application Data
(60) Provisional application No. 62/372,575, filed on Aug. 9, 2016.

(51) Int. Cl. A61F 6/12; A61F 2/00

(52) U.S. Cl. CPC A61F 6/12; A61F 2250/0029; A61F 2230/0045; A61F 2/005

(57) ABSTRACT

The present invention relates to an applicator for placing and positioning an object, such as a medical or veterinary device or treatment, within an orifice of a human or animal body. More specifically, the invention relates to an applicator for placing and positioning a pessary within the vagina and to kits comprising the applicator and a pre-loaded pessary. The invention also relates to methods of placing and positioning a pessary within the vagina and to methods for treating stress urinary incontinence in a woman in need thereof.

### RELATED APPLICATION

This application claims priority to U.S. Provisional Patent Application Ser. No. 62/372,575 filed on Aug. 9, 2016, the disclosure of which is incorporated by reference herein in its entirety.

### FIELD OF THE INVENTION

The present invention relates to an applicator for placing and positioning an object, such as a medical or veterinary device or treatment, within an orifice of a human or animal body. More specifically, the invention relates to an applicator for placing and positioning a pessary within the human vagina and to kits comprising the applicator and a pre-loaded pessary. The invention also relates to methods of placing and positioning a pessary within the human vagina and to methods for treating stress urinary incontinence in a woman in need thereof.

### BACKGROUND OF THE INVENTION

The insertion of various objects, such as medical or veterinary devices or treatments, into the orifice of a human or animal body is sometimes necessary for the treatment of the human or animal subject. The orifices into which there can be a need to place these objects can include the vagina, the anus, the urethra, the otic canal, the mouth, and the nasal cavities. Some examples of objects that one can have a need to place can include, for example, a pessary, a tampon, a tablet, a suppository, or a packing.

Urinary incontinence (UI) is a chronic condition which, combined with overactive bladder (OAB), affects a higher percentage of individuals in all age groups than do hypertension, depression or diabetes. The FY 2009 NIH Research Priorities for Women's Health published by NIH Office of Research on Women's Health (ORWH) states, “Basic, clinical and translational research should be considered in addressing priority areas in women's health research. Some examples may include, but are not limited to: . . . therapeutic interventions for acute and chronic diseases and disorders that affect women including . . . urologic diseases.” In 2007, the National Institute of Diabetes and Digestive and Kidney Diseases (NIDDK), along with other components of the NIH, sponsored a Consensus Development Program culminating in publication of National Institute of Health State-of-the-Science Conference Statement: Prevention of Fecal and Urinary Incontinence in Adults. The report states that, “Current national estimates are that more than 20 million women have urinary incontinence or have experienced it at some point in their lives.” This estimate is consistent with reported findings from other countries such as Canada, Holland, and Norway and suggests that 250-450 million women worldwide are affected by UI.

The NIH Conference Statement explores both economic and non-economic burdens of UI and concludes with the statement, “To reduce the suffering and burden of fecal and urinary incontinence, research is needed to establish underlying mechanisms, describe a classification system, determine natural history, classify persons according to their future risk for fecal or urinary incontinence, design interventions targeted to specific population groups, determine the effects of these interventions, and guide public policy.” This conclusion was echoed the following year in the Journal of the American Medical Association (JAMA) in an article on the results of the 2005-2006 National Health and Nutrition Examination Survey conducted by the National Centers for Health Statistics Centers for Disease Control and Prevention which ended by stating, “In conclusion, pelvic floor disorders affect a substantial proportion of women and increase with age . . . . Given the burden pelvic floor disorders place on US women and the health care system, research is needed to further understand their pathophysiology, prevention, and treatment.”

In the years since these reports, an emphasis has been placed on addressing a better understanding of pelvic floor disorders, and several new treatment devices and methods have been developed and refined. Among these are new versions of vaginal pessaries, which are devices that are inserted vaginally for management of various pelvic floor disorders including stress urinary incontinence. Development of these new pessaries has identified new requirements for their manufacturing, storage, deployment and positioning.

U.S. Pat. No. 8,323,176 B2, issued Dec. 4, 2012, to Spitz et al., and U.S. Pat. No. 7,981,021 B2, issued Jul. 19, 2011, to Spitz et al. disclose novel pessary devices and methods for the correction of stress urinary incontinence. The pessary devices disclosed therein have a horizontal proximal portion supported on the pubococcygeus muscle and a distal portion extending downward and away from the proximal portion at an angle approximating the vagina in an erect female as it passes down and between the anterior segments of the pubococcygeus muscles. The distal portion can terminate with a protrusion that is positioned to provide mid-urethral support, thereby preserving the urethrovesical angle.

When an object, such as a pessary, is placed within the vagina, there are several aspects of positioning that can be crucial for proper placement to maximize the object's intended effect. These aspects include, but are not limited to, the depth to which the object is inserted into the vagina, and the rotational positioning relative to the longitudinal axis of the vagina. For example, for pessary devices to provide their optimum benefit in managing stress urinary incontinence, it is important that the device be properly inserted and positioned within the vagina. Therefore, it would be desirable to have applicator devices for properly deploying, i.e. placing and positioning such objects, and particularly for pessaries.

It would also be desirable to have applicator devices in which the object to be inserted can be stored within the applicator device for an extended period of time prior to use without concern that being constrained within the applicator device can affect the functional shape of the object. Generally, tampons, vaginal tablets, and suppositories can be stored in applicator devices without concerns regarding undesired changes to their functional shape. But this is not always the situation. Furthermore, other objects, such as vaginal pessaries, are often compressed during the insertion process and should immediately return to their previous uncompressed shape when inserted. Such objects are sometimes made of a material that will be permanently deformed if stored for an extended period of time in an altered shape. Also, proper depth and rotational positioning are more likely to be an issue with vaginal pessaries, as contrasted with tampons, tablets, and suppositories.

It is apparent from the foregoing there is an unmet need for properly and conveniently deploying, in other words delivering and positioning objects, such as medical devices or treatments, within an orifice of the body of a human or animal subject. The present invention aims to provide an applicator for the deployment of such objects, and more specifically an applicator for placing and positioning a pessary within the human vagina. The present invention also aims to provide an applicator in which the pessary can be conveniently stored for an extended period without the concern that the applicator will affect the functional shape of the pessary. Also, the present invention aims to provide an applicator device that provides temporary compression of the pessary device during deployment allowing it to quickly return to its original uncompressed state when deployed, particularly when the pessary is made of a resilient material.

### BRIEF DESCRIPTION OF THE DRAWINGS

FIG. 0.1 is a perspective view of an embodiment of the applicator device as shown in FIG. 1.

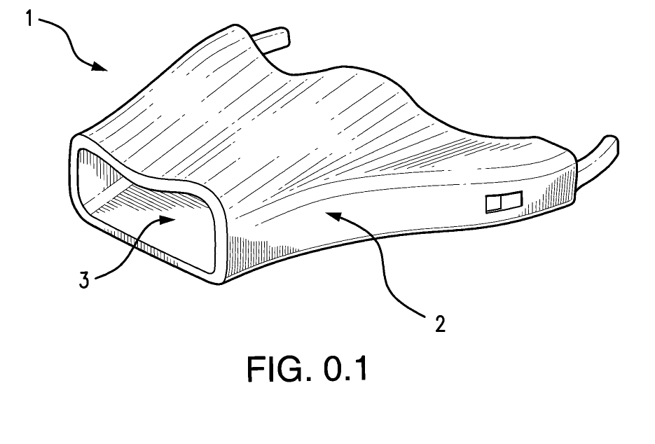

FIG. 0.2 is an exploded perspective view of FIG. 01 showing the applicator device with the pessary.

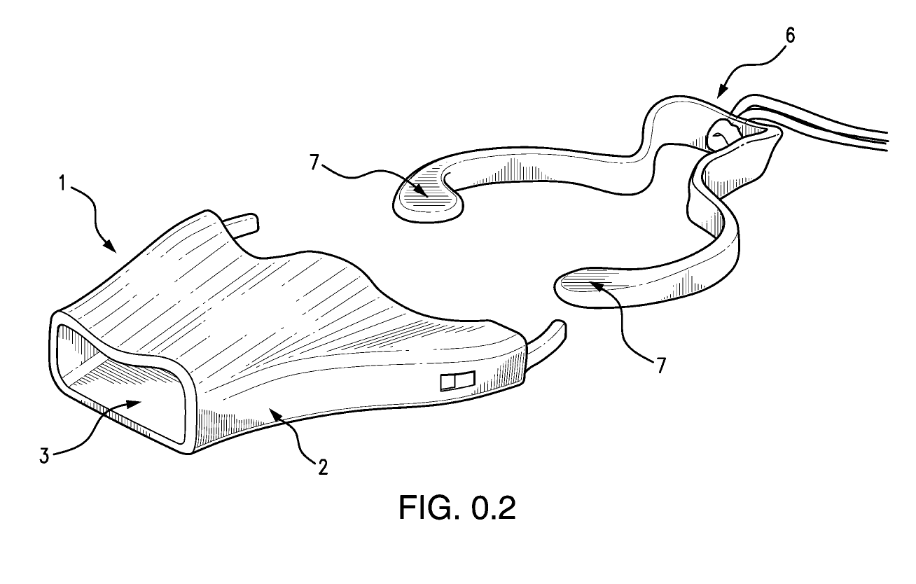

FIG. 1 is an embodiment of the invention demonstrating an incontinence pessary stored within the applicator device.

FIG. 1A is a planar view of the applicator device of FIG. 1 showing the longitudinal axis, as a dotted line.

FIG. 2 is a side view of the applicator of FIG. 1.

FIG. 3 is the applicator of FIG. 1 showing the pessary compressed during the process of deployment.

FIG. 4 is the applicator of FIG. 1 showing the pessary that has returned to its original uncompressed state near the end of deployment.

FIG. 5 is the applicator of FIG. 1 showing reinforcement of the narrower portion through which the pessary passes during deployment.

FIG. 6 is a side view of the applicator of FIG. 1 showing a longitudinal channel or groove on the inside (interior] of the applicator.

FIG. 7 is a cross-section of the widest portion of the applicator of FIG. 1 showing a longitudinal channel or groove on the inside (interior) of the applicator.

FIG. 7A is a cross-section of the widest portion of an alternative embodiment of the applicator of FIG. 1, showing longitudinal channels or groves on the inside (interior) surfaces, and also contours on one side of the applicator.

FIG. 8 is a side view of the applicator of FIG. 1 showing longitudinal protrusions on the inside (interior).

FIG. 9 is a cross-section of the widest portion of the applicator of FIG. 1 showing longitudinal protrusions on the inside (interior) of the applicator.

FIG. 9A is a cross-section of the widest portion of an alternative embodiment of the applicator of FIG. 1, showing longitudinal protusions on the inside (interior) surfaces of the applicator.

FIG. 9B is a cross-section of the widest portion of an alternative embodiment of the applicator of FIG. 1, showing a single longitudinal protrusion on an inside (interior) surface of the applicator.

FIG. 10 is a second embodiment of the invention demonstrating an incontinence pessary stored within the applicator.

FIG. 11 is a side view of the applicator of FIG. 10.

FIG. 12 is the applicator of FIG. 10 showing the pessary compressed during the process of deployment.

FIG. 13 is the applicator of FIG. 10 showing the pessary that has returned to its original uncompressed state near the end of deployment.

FIG. 14 is a third embodiment of the invention demonstrating an incontinence pessary stored within the applicator.

FIG. 15 is a side view of the applicator of FIG. 14.

FIG. 16 is the applicator of FIG. 14 showing the pessary compressed during the process of deployment.

FIG. 17 is the applicator of FIG. 14 showing the pessary that has returned to its original uncompressed state near the end of deployment.

FIG. 18 is a side view of the pelvis demonstrating placement of the applicator of FIG. 14.

### SUMMARY OF THE INVENTION

The present invention relates to an applicator for placing and positioning an object, such as a medical or veterinary device or treatment, within an orifice of a human or animal body. More specifically, the invention relates to an applicator for placing and positioning a pessary within the vagina and to kits comprising the applicator and a pre-loaded pessary. The invention also relates to methods of placing and positioning a pessary within the vagina and to methods for treating stress urinary incontinence in a woman in need thereof.

### DETAILED DESCRIPTION OF THE INVENTION

The present invention relates to an applicator device for placing and positioning a pessary within the vagina, comprising: an applicator body for receiving, transiting, and expelling the pessary, the applicator body having a hollow portion, interior and exterior surfaces, (a longitudinal axis with respect to the hollow portion of the body), a pessary receiving end (which is the distal end of the applicator body of the applicator), and a pessary expulsion end (which is the proximal end of the applicator body of the applicator and which terminates in an orifice), wherein the pessary receiving end and the pessary expulsion end are positioned oppositely to each other, wherein the pessary receiving end is dimensioned wider than the pessary expulsion end, wherein the pessary receiving end is dimensioned for receiving the pessary, and wherein the pessary expulsion end is dimensioned for insertion into the user's vaginal cavity to a predetermined depth, for compressing the pessary (to a desired degree of compression to enable its insertion in the vagina) during transit of the pessary from the pessary receiving end to the pessary expulsion end of the applicator body, and for expelling the pessary from the pessary expulsion end to effect the expansion, placement, and positioning of the pessary within the vagina.

In another aspect, the present invention relates to an applicator, wherein at least a portion of the pessary receiving end of the applicator body is dimensioned wider than the pessary expulsion end of the applicator body.

In another aspect, the present invention relates to an applicator, wherein the pessary receiving end of the applicator is dimensioned for directionally receiving the pessary and the pessary expulsion end of the applicator is dimensioned for directionally expelling the pessary.

In another aspect, the present invention relates to an applicator, wherein at least a portion of the pessary receiving end of the applicator body is dimensioned wider than the pessary expulsion end of the applicator body, such that the area that is dimensioned wider has a maximum width of about 3 cm to about 8 cm.

In another aspect, the present invention relates to an applicator, wherein the pessary receiving end of the applicator body further comprises a means for containing a pessary within the pessary receiving end.

In another aspect, the present invention relates to an applicator, wherein the means for containing a pessary within the pessary receiving end comprises an extension of the pessary receiving end of the applicator body that is dimensioned narrower than the widest portion of the pessary receiving end.

In another aspect, the present invention relates to an applicator, wherein the means for containing a pessary within the pessary receiving end of the applicator body comprises a flexible covering.

In another aspect, the present invention relates to an applicator, wherein the pessary receiving end of the applicator body further comprises a housing portion for containing the pessary in a noncompressed, partially compressed, or fully compressed form prior to transiting and expelling the pessary.

In another aspect, the present invention relates to an applicator, wherein the pessary receiving end of the applicator body further comprises a housing portion for containing the pessary in noncompressed form prior to transiting and expelling the pessary.

In another aspect, the present invention relates to an applicator, wherein the applicator further comprises a plunger for transiting and expelling the pessary through the applicator body of the applicator.

In another aspect, the present invention relates to an applicator, wherein the longitudinal axis with respect to the hollow portion of the applicator body of the applicator is a C-1 axis of symmetry, such that the applicator would have to be rotated 360 degrees around that axis to achieve its original appearance. In other words, this applicator would have no rotational symmetry.

In another aspect, the present invention relates to an applicator, wherein the longitudinal axis with respect to the hollow portion of the applicator body of the applicator is a C-2 axis of symmetry, such that the applicator could be rotated 180 degrees around that axis to be identical to achieve its original appearance. In other words, this applicator would have a two-fold axis of rotational symmetry.

In another aspect, the present invention relates to an applicator, wherein the applicator body of the applicator further comprises two opposing (essentially) flattened regions.

In another aspect, the present invention relates to an applicator, wherein the applicator body of the applicator further comprises two opposing contoured regions.

In another aspect, the present invention relates to an applicator, wherein the applicator body of the applicator further comprises an essentially flattened region and a contoured region, the flattened region being positioned oppositely to the contoured region.

In another aspect, the present invention relates to an applicator, wherein the applicator body of the applicator tapers from the pessary receiving end to the pessary expulsion end.

In another aspect, the present invention relates to an applicator, wherein the pessary expulsion end of the applicator body of the applicator is reinforced.

In another aspect, the present invention relates to an applicator wherein the proximal portion of the pessary expulsion end of the applicator body of the applicator (i.e., the end near the orifice) is cylindrical.

In another aspect, the present invention relates to an applicator, wherein the cylindrical portion of the pessary expulsion end of the applicator body of the applicator has an outer (overall) diameter from about 1 cm to about 5 cm and a longitudinal length from about 0.5 cm to about 10 cm.

In another aspect, the present invention relates to an applicator, wherein the cylindrical portion of the pessary expulsion end of the applicator body of the applicator has an outer diameter from about 1 cm to about 3.5 cm and a longitudinal length from about 0.5 cm to about 6 cm.

In another aspect, the present invention relates to an applicator, wherein the cylindrical portion of the pessary expulsion end of the applicator body of the applicator has an outer diameter from about 2 cm to about 3 cm and a longitudinal length from about 0.5 cm to about 3 cm.

In another aspect, the present invention relates to an applicator, wherein the proximal portion of the pessary expulsion end of the applicator body of the applicator is (i.e., the end near the orifice of the body of the applicator) ovoid.

In another aspect, the present invention relates to an applicator, wherein the proximal portion of the pessary expulsion end of the applicator body of the applicator is (i.e., the end near the orifice of the body of the applicator) oval.

In another aspect, the present invention relates to an applicator, wherein the oval portion of the pessary expulsion end of the applicator body of the applicator has an outer cross-sectional length of from about 1 cm to about 5 cm, an inner cross-sectional width from about 1 cm to about 5, and a longitudinal length from about 0.5 cm to about 10 cm.

In another aspect, the present invention relates to an applicator, wherein the oval portion of the pessary expulsion end of the applicator body of the applicator has an outer cross-sectional length of from about 1 cm to about 3.5 cm, an inner cross-sectional width from about 1 cm to about 3 cm, and a longitudinal length from about 0.5 cm to about 6 cm.

In another aspect, the present invention relates to an applicator, wherein the oval portion of the pessary expulsion end of the applicator body of the applicator has an outer cross-sectional length of from about 2 cm to about 3 cm, an inner cross-sectional width from about 1 cm to about 2 cm, and a longitudinal length from about 0.5 cm to about 3 cm.

In another aspect, the present invention relates to an applicator, wherein the proximal portion of the pessary expulsion end of the applicator body of the applicator (i.e., the end near the orifice of the body of the applicator) is rectangular with rounded edges (or one or more rounded/curved sides).

In another aspect, the present invention relates to an applicator, wherein the rectangular portion of the pessary expulsion end of the applicator body of the applicator has an outer cross-sectional length of from about 1 cm to about 5 cm, an inner cross-sectional width from about 1 cm to about 5, and a longitudinal length from about 0.5 cm to about 10 cm.

In another aspect, the present invention relates to an applicator, wherein the rectangular portion of the pessary expulsion end of the applicator body of the applicator has an outer cross-sectional length of from about 1 cm to about 3.5 cm, an inner cross-sectional width from about 1 cm to about 3 cm, and a longitudinal length from about 0.5 cm to about 6 cm.

In another aspect, the present invention relates to an applicator, wherein the rectangular portion of the pessary expulsion end of the applicator body of the applicator has an outer cross-sectional length of from about 2 cm to about 3 cm, an inner cross-sectional width from about 1 cm to about 2 cm, and a longitudinal length from about 0.5 cm to about 3 cm.

In another aspect, the present invention relates to an applicator, wherein the inner surface of the applicator body of the applicator comprises one or more (longitudinal) channels (or grooves).

In another aspect, the present invention relates to an applicator, wherein the inner surface of the applicator body of the applicator comprises two or more (longitudinal) channels (or grooves).

In another aspect, the present invention relates to an applicator, wherein the one or more (longitudinal) channels (or grooves) form a guidance means on the internal surface of the applicator body of the applicator.

In another aspect, the present invention relates to an applicator, wherein the inner surface of the applicator body of the applicator comprises one or more longitudinal protrusions.

In another aspect, the present invention relates to an applicator, wherein the one or more longitudinal protrusions form a guidance means on the internal surface of the applicator body of the applicator.

In another aspect, the present invention relates to an applicator, wherein the pessary is a device for temporary management of a medical condition selected from stress urinary incontinence, pelvic organ prolapse, or fecal incontinence.

In another aspect, the present invention relates to an applicator, wherein the medical condition is stress urinary incontinence.

In another aspect, the present invention relates to a kit for placing and positioning a pessary within the vagina, comprising:

(a) an applicator comprising an applicator body for receiving, transiting, and expelling the pessary, the applicator body having a hollow portion, interior and exterior surfaces, a longitudinal axis with respect to the hollow portion of the applicator body, a pessary receiving end (which is the distal end of the applicator body of the applicator), and a pessary expulsion end (which is the proximal end of the applicator body of the applicator and which terminates in an orifice), wherein the pessary receiving end and the pessary expulsion end are positioned oppositely to each other, wherein the pessary receiving end is dimensioned wider than the pessary expulsion end, wherein the pessary receiving end is dimensioned for receiving the pessary, and wherein the pessary expulsion end is dimensioned for insertion into the user's vaginal cavity to a predetermined depth, for compressing the pessary (to a desired degree of compression to enable the insertion of the pessary into the vagina) during transit of the pessary from the pessary receiving end to the pessary expulsion end of the applicator body, and for expelling the pessary from the pessary expulsion end to effect the expansion, placement, and positioning of the pessary within the vagina; and
(b) a pessary.

In another aspect, the present invention relates to a kit, wherein the pessary receiving end of the applicator body of the applicator further comprises a means for containing the pessary within the pessary receiving end.

In another aspect, the present invention relates to a kit, wherein at least a portion of the pessary receiving end of the applicator body of the applicator is dimensioned wider than the pessary expulsion end of the applicator body, wherein the pessary receiving end of the applicator body further comprises a housing portion containing the pessary in noncompressed form.

In another aspect, the present invention relates to a kit, wherein the pessary is contained within the housing portion of the pessary receiving end of the applicator body.

In another aspect, the present invention relates to a kit, wherein the pessary receiving end of the applicator body of the applicator is dimensioned for directionally receiving the pessary and the pessary expulsion end of the applicator body of the applicator is dimensioned for directionally expelling the pessary.

In another aspect, the present invention relates to a method for placing and positioning a pessary within the vagina, comprising inserting a pessary into the woman's vagina using the applicator device of the present invention and positioning the pessary so that when the woman is standing a portion of the pessary is supported by the woman's pubococcygeus muscle; a portion of the pessary descends between separated anterior fibers of the woman's pubococcygeus muscle; and a portion of the pessary is positioned to support the woman's urethra, preferably where the support is at a mid-urethral position.

In another aspect, the present invention relates to a method for treating stress urinary incontinence in a woman in need thereof, comprising; inserting a pessary into the woman's vagina using the applicator device of the present invention and positioning the pessary so that when the woman is standing a portion of the pessary is supported by the woman's pubococcygeus muscle; a portion of the pessary descends between separated anterior fibers of the woman's pubococcygeus muscle; and a portion of the pessary is positioned to support the woman's urethra, preferably where the support is at a mid-urethral position.

In another aspect, the present invention relates to an applicator for placing and positioning a device (or an object) within a human or animal cavity (a body cavity or orifice of the human or animal), comprising: an applicator body for receiving, transiting, and expelling the device, the applicator body having a hollow portion, interior and exterior surfaces, a longitudinal axis with respect to the hollow portion of the applicator body, a device receiving end (which is the distal end of the applicator body of the applicator), and a device expulsion end (which is the proximal end of the applicator body of the applicator and which terminates in an orifice, this orifice not to be confused with the human or animal cavity or orifice), wherein the device receiving end and the device expulsion end are positioned oppositely to each other, wherein the device receiving end is dimensioned wider than the device expulsion end, wherein the device receiving end is dimensioned for receiving the device, and wherein the device expulsion end is dimensioned for insertion into the human or animal cavity (orifice of the human or animal) to a predetermined depth, for compressing the device to a desired degree of compression to enable insertion of the device into the human or animal cavity (orifice of the human or animal) during transit of the device from the device receiving end to the device expulsion end of the applicator body, and for expelling the device from the device expulsion end to effect the expansion, placement, and positioning of the device within the human or animal cavity (or orifice).

In another aspect, the present invention relates to an applicator wherein the device receiving end of the applicator body of the applicator is dimensioned for directionally receiving the device and the device expulsion end of the applicator body of the applicator is dimensioned for directionally expelling the device.

In another aspect, the present invention relates to an applicator for placing and positioning a device within a human cavity (or orifice).

In another aspect, the present invention relates to an applicator, wherein the human cavity (or orifice) is selected from the vagina, the anus, the urethra, the otic (ear) canal, the mouth, or a nasal cavity.

In another aspect, the present invention relates to an applicator, wherein the human cavity (or orifice) is the vagina.

In another aspect, the present invention relates to an applicator, wherein the device or object is selected from a pessary, a tampon, a vaginal tablet, or a vaginal suppository.

In another aspect, the present invention relates to an applicator, wherein the pessary is a device for temporary management of a medical condition selected from stress urinary incontinence, pelvic organ prolapse, or fecal incontinence.

In another aspect, the present invention relates to an applicator, wherein the medical condition is stress urinary incontinence.

In another aspect, the present invention relates to a kit for placing and positioning a device (or an object) within a body cavity of a human or animal, comprising: (a) an applicator comprising an applicator body for receiving, transiting, and expelling the device, the applicator body having a hollow portion, interior and exterior surfaces, a device receiving end, and a device expulsion end, wherein the device receiving end and the device expulsion end are positioned oppositely to each other, wherein the device receiving end is dimensioned wider than the device expulsion end, wherein the device receiving end is dimensioned for receiving the device, and wherein the device expulsion end is dimensioned for insertion into the body cavity of the human or animal to a predetermined depth, for compressing the device during transit of the device from the device receiving end to the device expulsion end of the applicator body, and for expelling the device from the device expulsion end to effect the expansion, placement, and positioning of the device within the body cavity of the human or animal; and (b) a device.

These and other aspects of the invention will become apparent from the disclosure herein.

### Definitions

As used herein, the following terms have the indicated meanings unless expressly stated to the contrary:

The term “contoured” regions as used herein, means that the regions of the body of the applicator have a shape to them in those embodiments. This would be in contrast to “essentially flattened” regions.

The term “directionally” as used herein means that the object to which it refers has a defined orientation. The term also means that the object is moved or intended to be moved with a defined orientation. For example, the term “directionally receiving the pessary” means that the applicator device is designed to receive the pessary in a defined orientation and the term “directionally expelling the pessary” means that the applicator device expels the pessary in a defined orientation for proper placement and positioning within the vagina.

The term “distal” as used herein with respect to the applicator device means that portion of the applicator that is intended to receive the object or pessary or into which the object or pessary is inserted. The term distal is the opposite of the term proximal. The term “distal” with respect to the human female anatomy refers to the end of the vagina closest to the introitus and to the portion of the vaginal pessary that is intended to be oriented towards the distal end of the vagina.

The term “essentially” as used herein means that the parameter it defines does not have to be completely as such. For example, “essentially” flattened means “nearly, almost, or primarily” flattened.

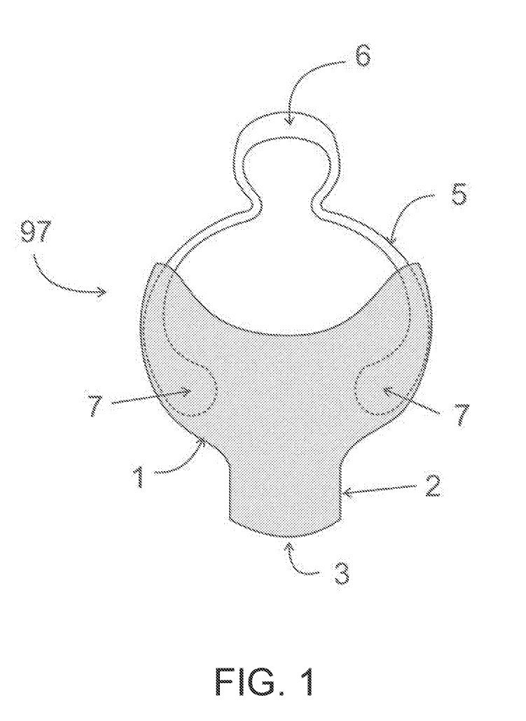

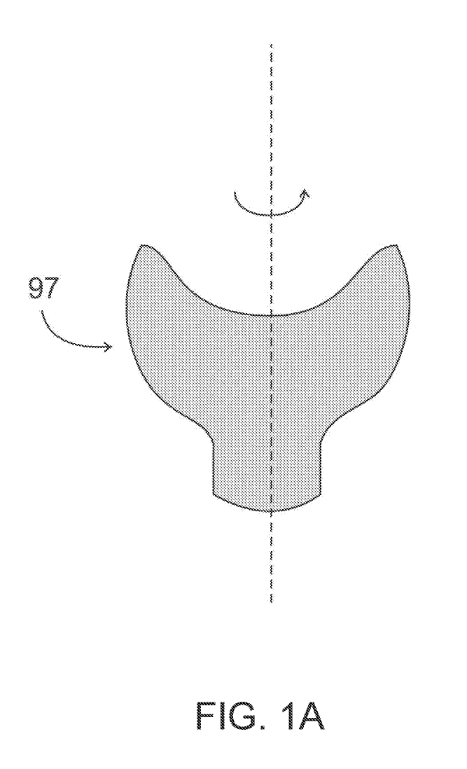

The term “longitudinal” as used herein, is with respect to and parallel or in the same direction as the axis of the body of the applicator, the axis being such that it defines an imaginary line through the body of the applicator. FIG. 1A shows a planar view of the applicator with the longitudinal axis depicted as a dotted line.

The term “proximal” as used herein with respect to the applicator device, means that portion of the applicator that is intended to expel the object or pessary or from which the object or pessary is expelled. This proximal portion of the applicator can be defined as terminating in or defining an orifice. The proximal portion of the applicator is the portion intended to be inserted into the body opening or cavity (an orifice) of the human or animal. The “orifice” of the applicator device is separate from and not intended to be confused with the “orifice” of the human or animal.

The term proximal, as used herein with respect to the human female anatomy refers to the end of the vagina closest to the cervix, and to the portion of the vaginal pessary that is intended to be oriented towards the proximal end of the vagina. The term “distal” refers to the end of the vagina closest to the introitus and to the portion of the vaginal pessary that is intended to be oriented towards the distal end of the vagina.

The term “reinforced” as used herein means that the applicator is thicker or has more material associated with it at that area of the applicator. This reinforcement can be achieved by a variety of means, including for example, overmolding the applicator at the desired area with a second material.

Applicators

As discussed above, it is an object of the invention to provide a solution for proper positioning of an item that is inserted into the vagina. It is a further object of the invention to provide a solution that allows the item to be stored in a manner that does not distort its shape during storage and which accomplishes temporary compression of the item during deployment, thus allowing it to quickly return to its uncompressed state, particularly when the item is made of a resilient material.

Disclosed herein are applicators (applicator devices) for inserting and positioning an item inside the vagina. The initial intended use is for inserting and positioning a vaginal pessary for temporary management of stress urinary incontinence. The applicators include a portion which can house a vaginal pessary prior to deployment and a portion which is inserted into the vagina. The portion which houses the pessary is of sufficient width that when it is aligned essentially parallel to the coronal plane of the body, it cannot be easily inserted into the vagina of the average woman without exerting significant force. The portion which is intended for insertion into the vagina is sufficiently narrow such that it can be inserted into the vagina of the average woman without exerting significant force. The force required for insertion is intended to be similar to that force that is required for insertion of commercially available tampon applicators. With the applicators of the present invention, when the narrower portion is inserted, the wider portion will control how deeply the applicator can be inserted into the vagina. The length of the narrower portion will control how deeply the pessary is inserted into the vagina when deployed. The two portions described connect with each other in a way that facilitates compression of the pessary during deployment and further facilitates proper passage of the pessary from the wider portion through the narrower portion during deployment of the pessary. The wider portion can have a length that is significantly less than its width. Such a shape can facilitate proper rotational orientation along the longitudinal axis of the applicator device due to the way that the average user would hold such a shaped applicator device. FIG. 1A is a planar view of the applicator showing the longitudinal axis of the device, represented as a dotted line. Additionally, features such as a groove or channel on the inside wall of the device, or a ridge or protuberance on the inside wall of the device, can help to guide the rotational positioning of the object being inserted.

In certain embodiments, the applicator device is comprised of an outer member which receives a separate inner member that acts as a plunger to push the pessary in a manner to expel it from the applicator during deployment. In other embodiments, the user can use a finger to expel the pessary from the applicator.

As discussed above, the term “proximal” with respect to the human female anatomy refers to the end of the vagina closest to the cervix, and to the portion of the vaginal pessary that is intended to be oriented towards the proximal end of the vagina. The term “distal” with respect to the human female anatomy refers to the end of the vagina closest to the introitus and to the portion of the vaginal pessary that is intended to be oriented towards the distal end of the vagina. In a normal, intact, female pelvis, the distal end of the vagina is constrained laterally by the pubococcygeus muscles, limiting the width of the vagina. Proximally, the vagina is not constrained laterally by the pubococcygeus muscles and in this area it can accommodate an object of greater width than can be accommodated in the distal vagina. Contraceptive diaphragms and several types of commercially available ring-shaped drug delivery devices are positioned in the wider proximal portion of the vagina when used properly. The present invention can be employed to aid insertion of various products (objects or devices) into the vagina. The invention is depicted here for use with a vaginal pessary for managing and treating stress urinary incontinence. This pessary has a certain shape and is made of a resilient material. During use of the pessary, the narrower, distal, portion of the pessary is intended to occupy a position in the distal portion of the vagina, underlying the urethra. The wider, proximal, portion of the pessary is intended to occupy a position further proximally where its width can be easily accommodated. Insertion of the pessary to the proper depth within the vagina is beneficial for maximum effectiveness of the pessary. See, U.S. Pat. No. 8,323,176 B2, issued Dec. 4, 2012, to Spitz et al., and U.S. Pat. No. 7,981,021 B2, issued Jul. 19, 2011, to Spitz et al., which are incorporated by reference herein in their entirety.

Insertion of the pessary into the vagina is facilitated by compression of the proximal arms of the pessary prior to, or during, the insertion process. After passing through the distal portion of the vagina, the proximal arms of the pessary are allowed to return to their original configuration and do so because of the resilient nature of the material from which the pessary can be constructed.

An applicator device as described herein facilitates the insertion of the pessary illustrated herein in several ways. A narrow portion of the applicator device can be inserted into the distal portion of the vagina where the opening of the narrow portion of the applicator device allows the pessary to be expelled from within the device into the appropriate location in the vagina. The length of this narrow portion can be in the range of 0.5 cm to 3 cm. This narrow portion of the applicator device is contiguous with a wide portion that cannot be easily inserted into the vagina of the average woman without exerting significant force. The widest area of this wide portion can be in the range of 3 cm to 8 cm. The illustrations herein depict an applicator device with a widest portion measuring approximately 5.5 cm. When the applicator device is inserted into the vagina using an amount of force that is similar to that force that is required for insertion of commercially available tampon applicators, the wide portion can cause insertion to stop. When the wide portion of the applicator device causes insertion to stop, the point at which the narrow portion is contiguous with the wide portion will be located approximately at the vaginal introitus. When the applicator device is in this position, the pessary can be deployed. When the pessary is pushed toward the vagina, the tapered shape of the inside of the wide portion of the applicator device facilitates compression of the resilient arms of the pessary, allowing the pessary to pass through the narrow end of the applicator device. When the pessary is pushed forward an additional amount, the arms of the pessary are expelled from the applicator device into the vagina. The applicator device is then removed from the vagina.

Applicators most commonly used for insertion of vaginal products have been cylindrically shaped tampon applicators that are longer in the longitudinal axis than in the horizontal axis. The current invention is such that a part, or the entirety, of the applicator device is a generally tubular structure that can house an object within the applicator device, and this tubular structure has at least one open end through which the object can be ejected. Note that in various embodiments of the present invention, the applicator comprises a housing or region where the device to be inserted can be stored in its essentially noncompressed form. The tubular structure can be configured in a way to create unique advantages over a cylindrical tampon applicator.

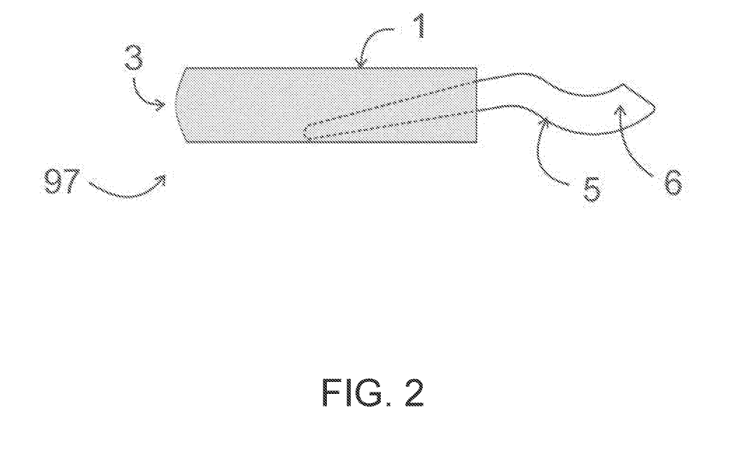

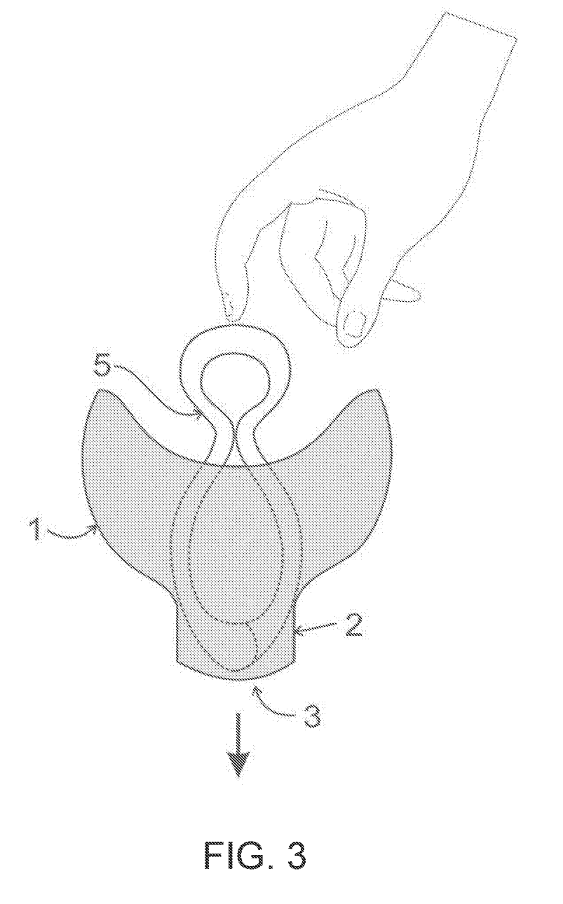

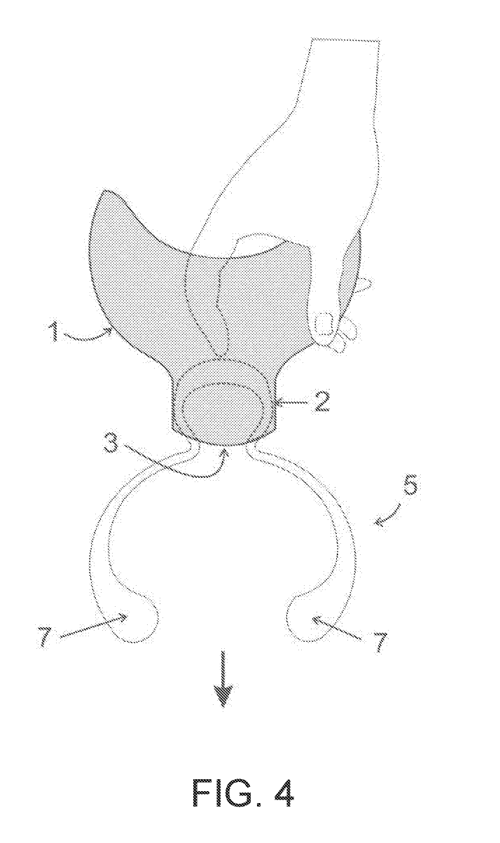

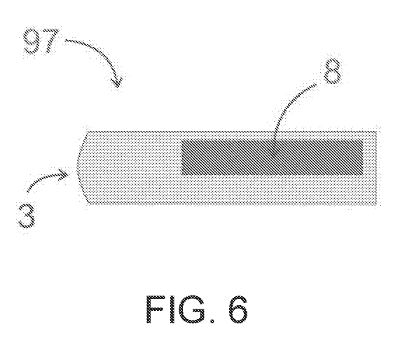

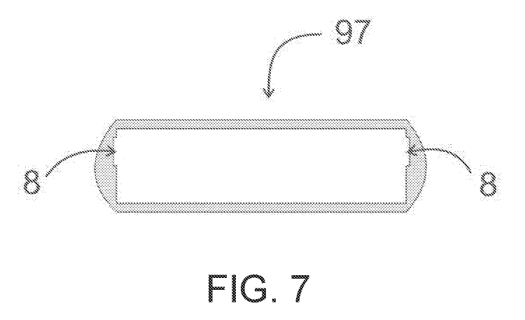

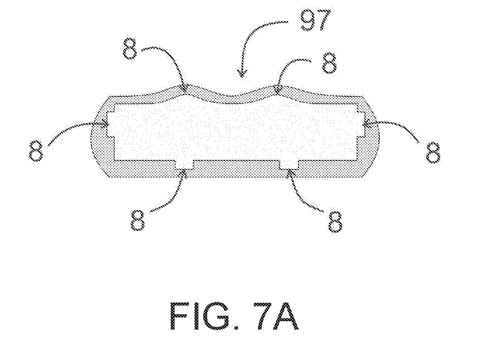

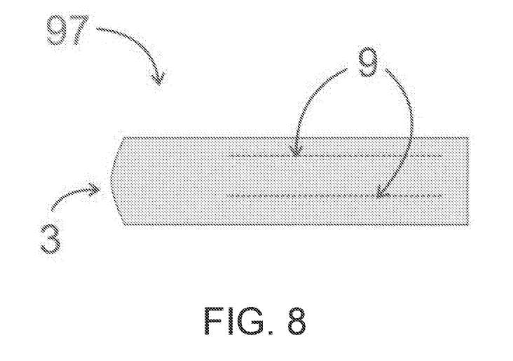

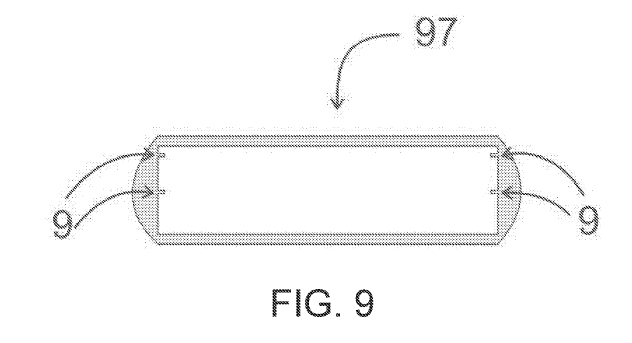

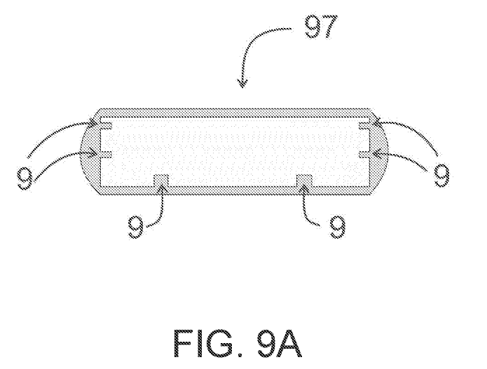

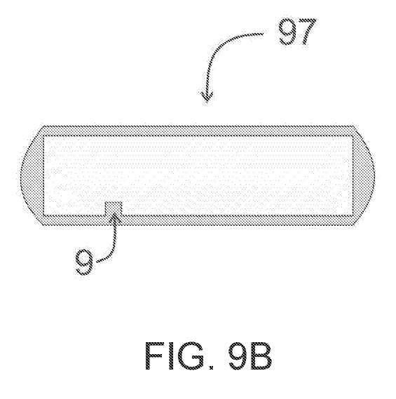

One embodiment of a device 97 is illustrated in top planar view in FIG. 1, FIG. 3, and FIG. 4, and in side view in FIG. 2, FIG. 6, and FIG. 8. A cross-section of the widest portion of the applicator device 97 is shown in FIG. 7 and FIG. 9. As illustrated in FIG. 1, the distal end 6 of the pessary 5 is oriented away from the narrow end 2 of applicator device 97 and the proximal arms 7 of the pessary 5 are oriented towards the narrow end 2 of applicator device 97. The pessary 5 is housed in its natural, uncompressed state with a portion of pessary 5 occupying the wide portion 1 of applicator device 97. Alternatively, the pessary 5 can be placed into the wide portion 1 when the user intends to deploy it. FIG. 3 and FIG. 4 illustrate how a finger is used to push the pessary 5 through the narrow open end 3 of applicator device 97 and into the vagina after applicator device 97 has been properly placed. FIG. 3 illustrates that the finger has pushed pessary 5 towards the narrow end 2 of applicator device 97. FIG. 3 further illustrates compression of the resilient arms 7 of the pessary 5 which has been facilitated by tapering of the inside of applicator device 97 from its area of widest cross-section 1 towards its narrow portion 2. In FIG. 3, the compressed resilient arms 7 of the pessary 5 have entered the narrow portion 2 of the applicator device 97. FIG. 4 illustrates almost complete deployment of pessary 5. FIG. 4 further illustrates that the resilient arms 7 of the pessary 5 have exited the open end 3 of applicator device 97 and have returned to their original uncompressed state.

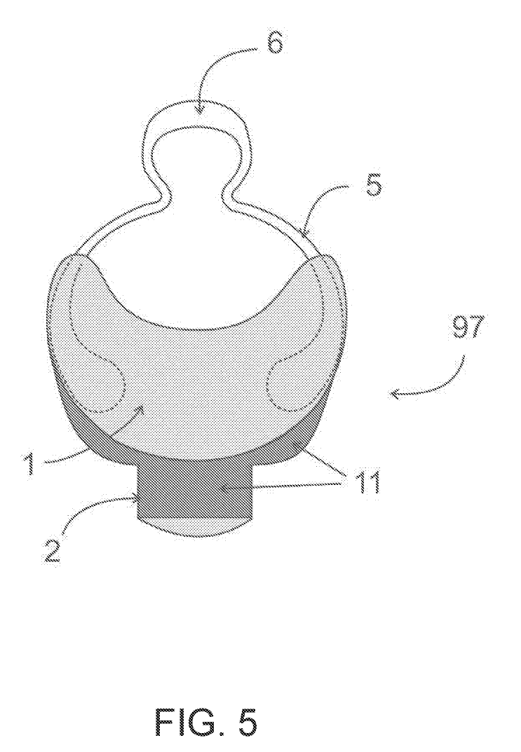

FIG. 5 illustrates reinforcement of an area 11 of applicator device 97. FIG. 5 illustrates the reinforced area 11 as including a segment of the wide portion 1 of applicator device 97 and all of the narrow portion 2 of applicator device 97. Alternatively, the reinforced area 11 can include only a segment of the narrow portion 2, or it can include a segment or all of the narrow portion 2 and none of the wide portion 1. Reinforcement of the reinforced area 11 can be accomplished by thickening the walls of applicator device 97 in the reinforced area 11, or by addition of another material as an overlay or insert. FIG. 5 further illustrates that the inside wall of applicator device 97 and the outside wall of applicator device 97 may not be parallel. A specific tapered configuration of the inside wall of applicator device 97 as it transitions from the wider portion 1 to the narrower portion 2 of applicator device 97 can be beneficial for optimal compression of the pessary 5 as it is deployed. A different tapered configuration of the outside wall of applicator device 97 can be desirable to reliably limit how far the applicator device 97 can be inserted into the vagina.

FIG. 6 and FIG. 7 illustrate a groove 8 or channel 8 on the inside of applicator device 97. The groove 8 or channel 8 is of an appropriate size to accommodate the resilient arms 7 of pessary 5 to guide pessary 5 through applicator device 97 during deployment of the pessary 5.

Similarly, FIG. 8 and FIG. 9 illustrate protuberances (i.e. protrusions) 9 on the inside of applicator device 97. The protuberances 9 are an appropriate size and spacing to accommodate the resilient arms 7 of pessary 5 to guide pessary 5 through applicator device 97 during deployment of the pessary 5. These protuberances guides can help maintain proper rotational positioning of the pessary 5 during deployment.

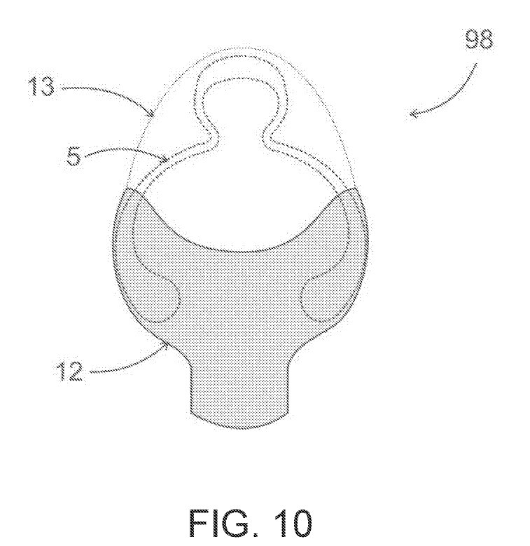

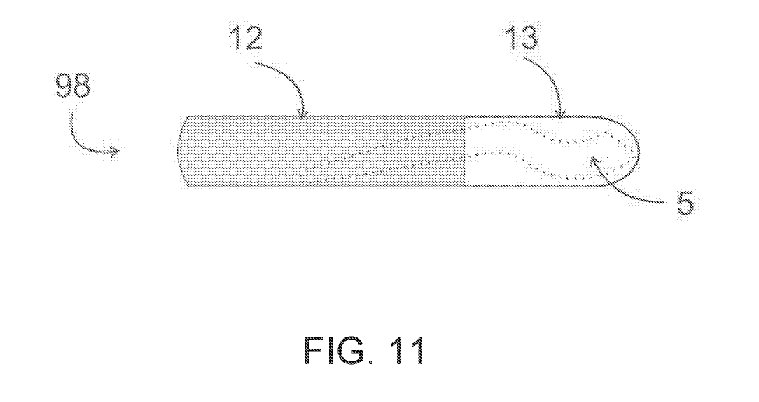

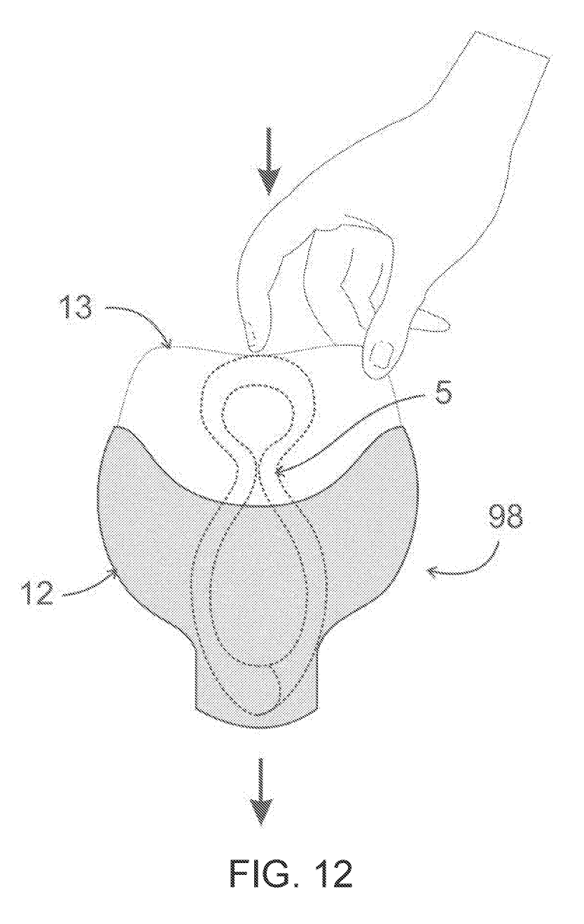

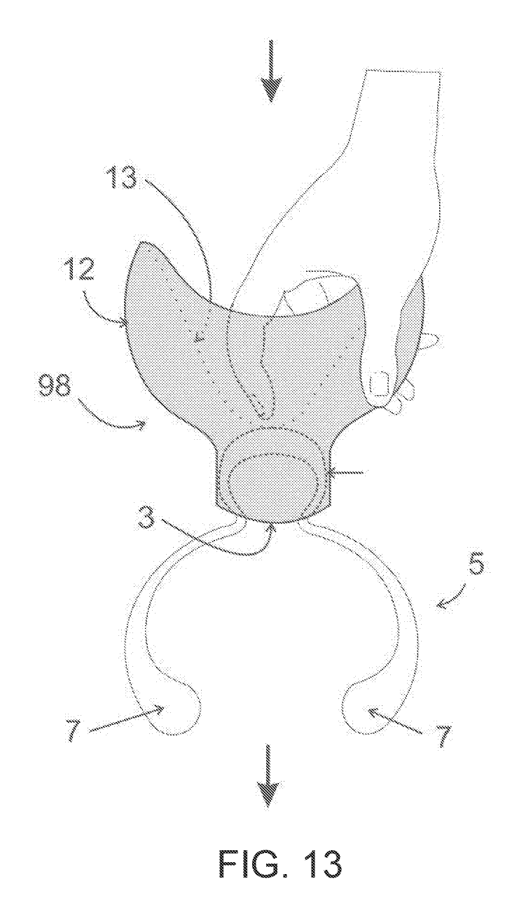

Another embodiment of an applicator device 98 is illustrated in top planar view in FIG. 10, FIG. 12, and FIG. 13, and in side view in FIG. 11. As with applicator device 97, a finger is used to deploy pessary 5 with applicator device 98, as illustrated in FIG. 12 and FIG. 13. Applicator device 98 is constructed of 2 separate materials, as illustrated in FIG. 10, FIG. 11, FIG. 12, and FIG. 13. The proximal end of pessary 5 is housed within a portion 12 of applicator device 98 sufficiently strong to maintain its general shape throughout the process of deploying pessary 5. This portion 12 can be constructed of various materials such as a cardboard material or molded paper slurry. Preferentially, this portion 12 is constructed of a plastic resin. The distal end of pessary 5 is housed within a portion 13 of applicator device 98 that is constructed of a flexible material, such as paper, and this portion 13 is deformed by the finger that is used to deploy the pessary 5. This applicator device 98 either can or does not, depending on the desired embodiment, include the groove 8, protuberances 9, and reinforcement 11 as described above and illustrated with applicator device 97. FIG. 12 illustrates an interim step during the deployment of pessary 5 and illustrates deformation of portion 13 by the finger. FIG. 13 illustrates almost complete deployment of pessary 5. Portion 13 is not visible in FIG. 13 as it has been pushed entirely inside portion 12 by the finger.

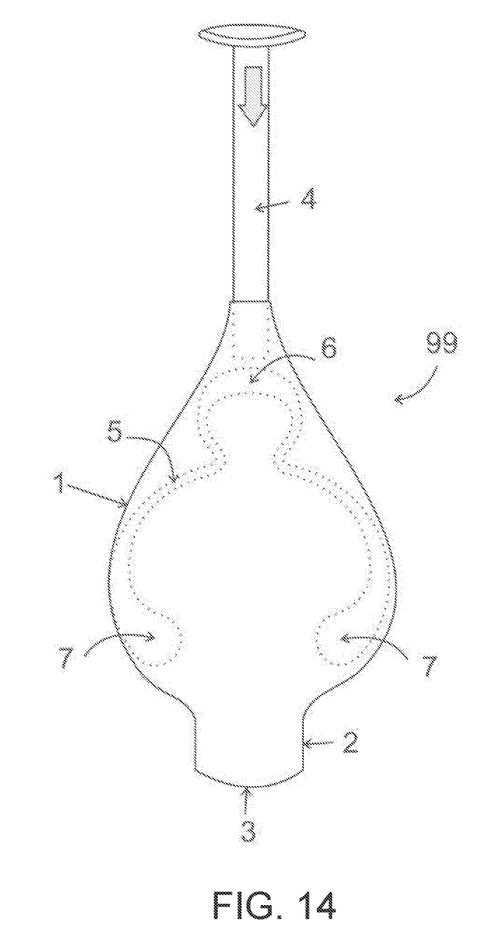

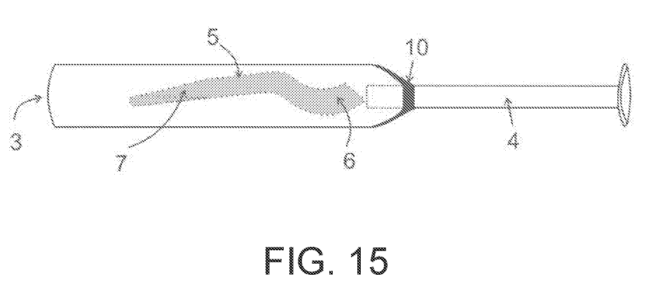

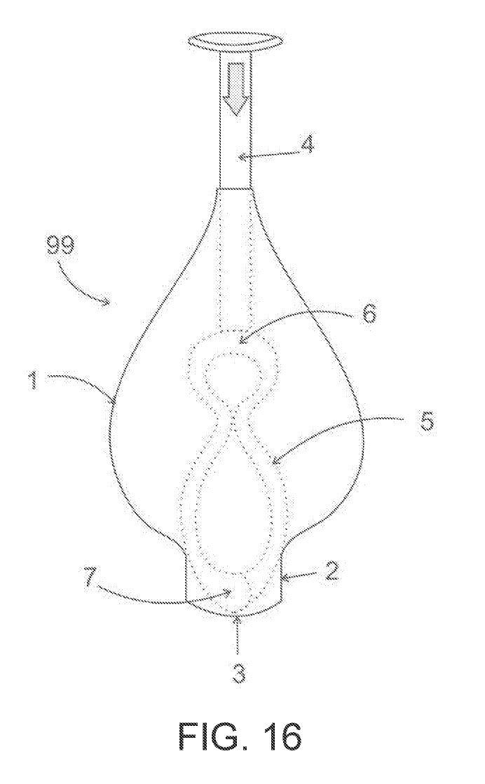

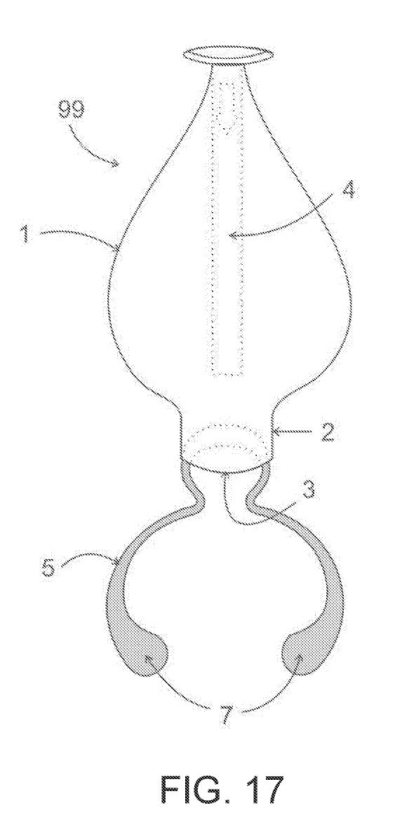

An additional embodiment of an applicator device 99 is illustrated in top planar view in FIG. 14, FIG. 16, and FIG. 17, and in side view in FIG. 15. The shape and position of a vaginal pessary 5 are illustrated when stored within the applicator device 99 and during deployment of the pessary 5. In this embodiment of applicator device 99, a plunger 4 is illustrated. This plunger 4 is used instead of a finger to push the pessary 5 through the narrow open end 3 of applicator device 99 and into the vagina after applicator device 99 has been properly placed.

FIG. 15 illustrates a thickening 10 of the wall of applicator device 99 where it receives a plunger 4. This thickening can help to stabilize plunger 4 during deployment of the pessary 5.

In FIG. 14 and FIG. 15 the pessary 5 is illustrated housed inside applicator device 99 in its position during storage. As with applicator device 97 and applicator device 98, the distal end 6 of the pessary 5 is oriented away from the narrow end 2 of applicator device 99 and the proximal arms 7 of the pessary 5 are oriented towards the narrow end 2 of applicator device 99. The pessary 5 is housed in its natural, uncompressed state. FIG. 16 illustrates an interim step in the deployment of the pessary 5. In FIG. 16, the plunger 4 has been pushed partially into the wide portion 1 of the applicator device 99. This has pushed the pessary 5 towards the open end 3 of the applicator device 99. FIG. 17 illustrates almost complete deployment of pessary 5. In FIG. 17, the plunger 4 is shown to be fully depressed into applicator device 99. FIG. 17 further illustrates that the resilient arms 7 of the pessary 5 have exited the open end 3 of applicator device 99 and have returned to their original uncompressed state. This applicator device 99 either can or does not include, depending on the desired embodiment, the groove 8, protuberances 9 and reinforcement 11 as described above and illustrated with applicator device 97.

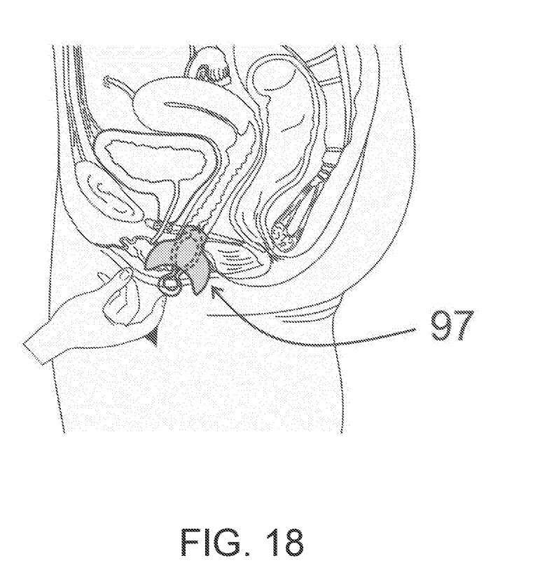

FIG. 18. illustrates applicator device 99 when it has been properly placed in the vagina. For illustration purposes, the applicator device 99 that is illustrated in FIG. 18 is aligned generally along the sagittal plane of the body, but during actual use, it may be preferentially aligned generally along the coronal plane of the

Kits

As described above, in another aspect, the present invention relates to a kit for placing and positioning a pessary within the vagina. These kits contain the applicator device of the present invention and the pessary. The pessary can be provided in pre-loaded or stored form within the housing of the applicator device. The kits comprise: (a) an applicator comprising an applicator body for receiving, transiting, and expelling the pessary, the applicator body having a hollow portion, interior and exterior surfaces, a longitudinal axis with respect to the hollow portion of the applicator body, a pessary receiving end (which is the distal end of the body of the applicator), and a pessary expulsion end (which is the proximal end of the body of the applicator and which terminates in an orifice), wherein the pessary receiving end and the pessary expulsion end are positioned oppositely to each other, wherein the pessary receiving end is dimensioned wider than the pessary expulsion end, wherein the pessary receiving end is dimensioned for directionally receiving the pessary, and wherein the pessary expulsion end is dimensioned for insertion into the user's vaginal cavity to a predetermined depth, for compressing the pessary (to a desired degree of compression to enable its insertion in the vagina) during transit of the pessary from the pessary receiving end to the pessary expulsion end of the applicator body, and for directionally expelling the pessary from the pessary expulsion end to effect the expansion, placement, and positioning of the pessary within the vagina; and (b) a pessary.

Methods of Placement and Positioning

The proper insertion, i.e. placement and positioning, of the pessary is important for providing management or treatment of stress urinary incontinence. The present invention relates to a method for placing and positioning a pessary within the vagina, comprising inserting a pessary into the woman's vagina using the applicator device of the present invention and positioning the pessary so that when the woman is standing a portion of the pessary is supported by the woman's pubococcygeus muscle; a portion of the pessary descends between separated anterior fibers of the woman's pubococcygeus muscle; and a portion of the pessary is positioned to support the woman's urethra, preferably where the support is at a mid-urethral position.

Methods of Treating Stress Urinary Incontinence

The present invention also relates to methods of treating stress urinary incontinence, which is due to insufficient strength of the closure of the bladder. Such methods comprise; inserting a pessary into the woman's vagina using the applicator device of the present invention and positioning the pessary so that when the woman is standing a portion of the pessary is supported by the woman's pubococcygeus muscle; a portion of the pessary descends between separated anterior fibers of the woman's pubococcygeus muscle; and a portion of the pessary is positioned to support the woman's urethra, preferably where the support is at a mid-urethral position.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the present invention, where the term comprises is used with respect to the recited component or steps, it is also contemplated that the present invention consists essentially of, or consists of, the recited components or steps. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

1. An applicator device for placing and positioning a pessary within the vagina, comprising:
an applicator body for receiving, transiting, and expelling the pessary, the applicator body having a hollow portion, interior and exterior surfaces, a pessary receiving end, and a pessary expulsion end,
wherein the pessary receiving end and the pessary expulsion end are positioned oppositely to each other,
wherein the pessary receiving end is dimensioned wider than the pessary expulsion end,
wherein the pessary receiving end is dimensioned for receiving the pessary, and wherein the pessary expulsion end is dimensioned for insertion into the user's vaginal cavity to a predetermined depth, for compressing the pessary during transit of the pessary from the pessary receiving end to the pessary expulsion end of the applicator body, and for expelling the pessary from the pessary expulsion end to effect the expansion, placement, and positioning of the pessary within the vagina.

2. An applicator according to claim 1 wherein at least a portion of the pessary receiving end of the applicator body is dimensioned wider than the pessary expulsion end of the applicator body.

3. An Applicator according to claim 2 wherein the pessary receiving end is dimensioned for directionally receiving the pessary and the pessary expulsion end is dimensioned for directionally expelling the pessary.

4. An applicator according to claim 3 wherein the pessary receiving end of the applicator body further comprises a means for containing a pessary within the pessary receiving end.

5. (canceled)

6. An applicator according to claim 4 wherein the means for containing a pessary within the pessary receiving end of the applicator body comprises a flexible covering.

7. An applicator according to claim 3 wherein the pessary receiving end of the applicator body further comprises a housing portion for containing the pessary in a noncompressed, partially compressed, or fully compressed form prior to transiting and expelling the pessary.

8. (canceled)

9. An applicator according to claim 7 wherein the applicator further comprises a plunger for transiting and expelling the pessary through the applicator body of the applicator.

10.-12. (canceled)

13. An applicator according to claim 3 wherein the applicator body tapers from the pessary receiving end to the pessary expulsion end.

14.-21. (canceled)

22. An applicator according to claim 3 wherein the inner surface of the applicator body comprises one or more channels.

23. (canceled)

24. An applicator according to claim 22 wherein the one or more channels form a guidance means on the internal surface of the applicator body.

25. An applicator according to claim 3 wherein the inner surface of the applicator body comprises one or more protrusions.

26. An applicator according to claim 25 wherein the one or more protrusions form a guidance means on the internal surface of the applicator body.

27. An applicator according to claim 1 wherein the pessary is a device for temporary management of a medical condition selected from stress urinary incontinence, pelvic organ prolapse, or fecal incontinence.

28. An applicator according to claim 27 wherein the medical condition is stress urinary incontinence.

29. A kit for placing and positioning a pessary within the vagina, comprising:
(a) an applicator comprising an applicator body for receiving, transiting, and expelling the pessary, the applicator body having a hollow portion, interior and exterior surfaces, a pessary receiving end, and a pessary expulsion end,
wherein the pessary receiving end and the pessary expulsion end are positioned oppositely to each other,
wherein the pessary receiving end is dimensioned wider than the pessary expulsion end,
wherein the pessary receiving end is dimensioned for receiving the pessary, and wherein the pessary expulsion end is dimensioned for insertion into the user's vaginal cavity to a predetermined depth, for compressing the pessary during transit of the pessary from the pessary receiving end to the pessary expulsion end of the applicator body, and for expelling the pessary from the pessary expulsion end to effect the expansion, placement, and positioning of the pessary within the vagina; and
(b) a pessary.

30.-33. (canceled)

34. A method for placing and positioning a pessary within the vagina, comprising inserting a pessary into the woman's vagina using the applicator device of claim 1 and positioning the pessary so that when the woman is standing a portion of the pessary is supported by the woman's pubococcygeus muscle; a portion of the pessary descends between separated anterior fibers of the woman's pubococcygeus muscle; and a portion of the pessary is positioned to support the woman's urethra.

35. A method for treating stress urinary incontinence in a woman in need thereof, comprising; inserting a pessary into the woman's vagina using the applicator device of claim 1 and positioning the pessary so that when the woman is standing a portion of the pessary is supported by the woman's pubococcygeus muscle; a portion of the pessary descends between separated anterior fibers of the woman's pubococcygeus muscle; and a portion of the pessary is positioned to support the woman's urethra.

36. An applicator device for placing and positioning a device within a body cavity of a human or animal, comprising:
an applicator body for receiving, transiting, and expelling the device, the applicator body having a hollow portion, interior and exterior surfaces, a device receiving end, and a device expulsion end,
wherein the device receiving end and the device expulsion end are positioned oppositely to each other,
wherein the device receiving end is dimensioned wider than the device expulsion end,
wherein the device receiving end is dimensioned for receiving the device, and wherein the device expulsion end is dimensioned for insertion into the body cavity of the human or animal to a predetermined depth, for compressing the device during transit of the device from the device receiving end to the device expulsion end of the applicator body, and for expelling the device from the device expulsion end to effect the expansion, placement, and positioning of the device within the body cavity of the human or animal.

37. An applicator according to claim 36 wherein the body cavity is selected from the vagina, the rectum, the urethra, the mouth, the ear canal, and a nasal cavity.

38. An applicator according to claim 36 for placing and positioning a device within the body cavity of a human.

39.-45. (canceled)
